(19) United States
(12) Patent Application Publication
(10) Pub. No.: US 2002/0009765 A1
(43) Pub. Date: Jan. 24, 2002

(54) METHOD FOR ASSESSING VIABILITY OF LIVE MICROBIAL THERAPEUTIC AGENTS

(76) Inventors: ENG-HONG LEE, GUELPH (CA); TAPASHI DASGUPTA, GUELPH (CA)

Correspondence Address: JOHN C JEFFREY, DENNISON ASSOCIATES, SUITE 301, 133 RICHMOND STREET WEST, TORONTO, M5H2L7, CA

(21) Appl. No.: 09/140,727

(22) Filed: Aug. 27, 1998

(30) Foreign Application Priority Data
Aug. 29, 1997 (CA) 2,214,233

(51) Int. Cl. C12Q 1/02

(52) U.S. Cl. 435/29

(57) ABSTRACT

The present invention provides for a method of assessing for viability of organisms in a live microbial therapeutic preparations. The method involves first measuring a suspension of the organisms for the quantity of a selectable marker indicative of viable organisms. The measurements are then compared against a standard curve to give a count of the viable organisms in the suspension. In a preferred embodiment, the present invention is directly a method of assessing the viability of Eimeria oocysts in a live coccidiosis vaccine. The method involves first measuring a suspension of the Eimeria oocysts for the quantity of ATP bioluminescence in the suspension. The quantity of ATP bioluminescence in the suspension may be compared against a predetermined standard to give a count of the viable Eimeria oocysts in the live coccidiosis vaccine.

### FIELD OF THE INVENTION

The present invention relates to a method for assessing viability of organisms in a live microbial therapeutic agent, and in particular, a method for assessing the viability of organisms in a live vaccine or probiotic.

### BACKGROUND OF THE INVENTION

There are many disease states which are prevented or controlled through the use of live microbial therapeutic agents. One such example is the use of live attenuated vaccines to immunize animals against a disease state caused by a microbial organism. When administering the vaccine, it is important to know the titer of the vaccine or the number of viable organisms present in the vaccine. When the vaccines are freshly prepared, it is relatively simple to formulate the vaccine to have the required number of viable organisms for proper treatment. However, with storage of the vaccine, even under recommended conditions, the viability of the organisms gradually decreases. If the vaccine is stored under potentially detrimental conditions or if stored for long periods of time, the viability of the organisms may decrease to a level which may not provide adequate protection against the disease state. This is particularly important for vaccines which utilize low numbers of organisms or where the storage conditions may not be ideal.

For example, at the present time, poultry hatchlings, within the first few days of life, are required to be immunized against various diseases and the type of vaccine used for each disease dictates its method of administration. Live vaccines are commonly administered once the hatchlings are established in their brooding trays in the form of aqueous suspensions either sprayed on feed or added to the drinking water.

One example of a live vaccine is that used to immunize poultry against coccidiosis caused by protozoa of the genus Eimeria. Coccidiosis is a very common disease of poultry and there are several species of Eimeria which are known to cause such disease. The symptoms and severity of the disease are dependent upon the species of Eimeria with which the bird is infected with E. tenella, E. acervulina and E. maxima being three of the most prevalent species. Coccidiosis vaccines are at present comprised of virulent strains of coccidia in a suitable carrier for administration, the coccidia being capable of causing a mild form of the disease and selected to be very anticoccidial susceptible. Generally, the coccidia vaccines contain small numbers of organisms, as few as 200 or less organisms per bird to be immunized. Hatcheries are generally located great distances from the places of formulation of the vaccines and thus uncertain transportation may be required to provide the vaccine to the hatchery. In addition, the storage conditions at hatcheries may not be ideal and there may not be facilities for maintaining the vaccine at the proper storage temperature. All of these factors may result in the birds not being properly immunized if a vaccine which has not been stored properly is utilized. It may also be necessary to discard otherwise usable vaccine if it has been stored under questionable conditions. Up to the present time, the only method for determining viability of Eimeria is through infectivity or lesion scores in chicks, which may take 5 to 7 days to complete and requires a skilled person to perform the assay and interpret the results.

There are many other examples of live vaccines for which the only method for determining viability is through plate counts. Once again, such methods for determining viability takes several days to obtain the results and they are not easily adaptable for use outside of a laboratory.

Another example of a live microbial therapeutic agent utilized in prevention of a disease state are probiotics or competitive exclusion products, which are generally defined as microbial cultures which have a beneficial effect on the animal to which they are administered. These agents are particularly utilized to populate the intestinal tract of an animal to aid in preventing disease causing organisms from being able to be established in the intestinal tract of the animal. Probiotics are administered to a number of different animals in order to provide for population or repopulation of the normal intestinal flora in the animal. For example, in cattle, administration of Lactobacillus acidophilus helps increase nutrient absorption efficiency and controls the proliferation of potentially harmful microorganisms that could cause disease states that may adversely affect development and weight gain. The use of probiotics in cattle can help restore optimal intestinal flora especially after stressful situations such as transport.

Recently, the use of probiotics for control of Salmonella infections in poultry has been proposed. The probiotics for such use generally contain a number of different species and strains of microorganisms isolated from the normal intestinal flora of adult poultry. Examples of such probiotic preparations are given in U.S. Pat. Nos. 4,689,226, 5,478,557, and 5,308,615 among others. The microorganisms generally utilized in the probiotic are anaerobic bacteria which are adversely sensitive to environmental influences such as oxygen, moisture, temperature extremes and many chemicals. The probiotics are generally provided as a lyophilized preparation which must be reconstituted before use and used immediately. The viability of the probiotic may be reduced because of the storage and handling conditions to which the lyophilized preparations have been exposed. However, until the preparation is reconstituted, it is not possible to know the viable count of the preparation. Even then, it is difficult if not impossible, especially in the field, to obtain a determination of the viability in sufficient time to adjust dosage levels.

It would therefore be useful to have a simple method for determining the viability of the organisms in a live microbial therapeutic agent such as a vaccine or probiotic prior to administration, such that if necessary the dosage levels may be adjusted.

### SUMMARY OF THE INVENTION

The present invention provides for a method of assessing for viability of organisms in a live microbial therapeutic preparation. The method comprises:

a) measuring a suspension of the organisms for the quantity of a selectable marker indicative of viable organisms; and

b) comparing the measurements of step a) against a standard curve to give a count of the viable organisms in the suspension.

In a preferred embodiment, the present invention is directed to a method of assessing the viability of Eimeria oocysts in a live coccidiosis vaccine. The method comprises:

a) measuring a suspension of the Eimeria oocysts for the quantity of ATP bioluminescence in the suspension; and

b) comparing the quantity of ATP bioluminescence in the suspension of step a) against a standard curve to give a count of the viable Eimeria oocysts in the live coccidiosis vaccine.

In an aspect of the invention, there is provided a method of providing an optimal number of viable organisms in a live microbial therapeutic preparation. The method comprises:

a) measuring a suspension of the organisms for the quantity of a selectable marker indicative of viable organisms;

b) comparing the measurements of step a) against a standard curve to give a count of the viable organisms in the suspension; and

c) adjusting the volume of the suspension of organisms or the dosage of the suspension to be administered to provide an optimal number of viable organisms in the dosage to be administered.

In another aspect of the invention there is provided a kit for assessing the number of viable organisms in a live microbial therapeutic preparation in the field. The kit comprises reagents necessary for performing the test for a selectable marker indicative of viable organisms, a means of measuring the result of the test to determine the level of the selectable marker in the test sample and a means for determining the number of viable organisms present based on the level of the selectable marker.

In yet another aspect of the invention there is provided a kit for assessing viability of Eimeria oocysts in a live coccidiosis vaccine in the field by the measurement of the ATP levels using bioluminescence, the kit comprising test reagent for dilution of the sample, an enzyme reagent, and a means for determining the number of viable organisms present based on the ATP level.

### BRIEF DESCRIPTION OF THE DRAWINGS

Preferred embodiments of the present invention are illustrated in the drawings in which:

FIG. 1 is an X-Y scatter plot of the log of relative light units (RLU) of mixed baterial samples versus log of viable plate counts; and

FIG. 2 is an X-Y scatter plot of the log of predicted viable count of mixed baterial samples versus log of calculated(plate) counts.

### DETAILED DESCRIPTION OF THE PREFERRED EMBODIMENTS

There are many disease states, particularly in animal husbandry which are controlled or prevented by live microbial therapeutic preparations. The present invention is directed in one aspect to a method of assessing the viability of live microbial therapeutic preparations in the field, prior to their administration. Traditionally, assessing viability of live microbial therapeutic preparations has involved testing which can take several days and can require highly skilled workers to complete. The present invention provides for a method of assessing viability of the organisms in a live microbial therapeutic preparations which is easily adaptable to use by low skilled workers.

The present invention is based on the measurement of a selectable marker which is indicative of viability of the organisms in the live microbial therapeutic preparations. The selectable marker chosen is one which is relatively uniform in the organism of the microbial therapeutic preparations and which is easily measured accurately so that the values of the measurement may be compared to a standard curve to give an indication of the number of viable organisms in the microbial therapeutic preparations. Once the number of viable organisms in the microbial therapeutic preparations is known, the volume of the suspension of organisms or the dosage of the microbial therapeutic preparations may be adjusted if necessary.

While there are a number of selectable markers which can be utilized in practicing the present invention, such as the levels of nucleotides including adenosine triphosphate (ATP), adenosine diphosphate (ADP), flavin mononucleotide (FMN), nicotinamide adenine dinucleotide (NAD) its phosphorylated and reduced derivatives, and many others, the measurement of ATP levels in the organism is preferred.

Firefly bioluminescence measurement of ATP has been described in a number of references including U.S. Pat. Nos. 3,745,090, 3,941,703, 3,933,592, and 4,246,340 as a rapid and sensitive method for determining the number of viable bacterial cells in a sample. In this method, ATP reacts with luciferase enzyme and luciferin substrate purified from firefly light organs in the presence of magnesium catalyst and molecular oxygen producing photons in the yellow/green range of the visible light spectrum. The photon efficiency of the reaction is 85% or greater, and since photons can be counted down to a few photons per second, the reaction offers an extremely sensitive method of assaying ATP. Utilizing purified luciferase enzyme the reaction is specific to ATP and other nucleotides do not interfere with the measurement so long as there are no enzymes in the sample to convert other nucleotides to ATP. The quantity of measured ATP may be converted to the number of cells by dividing the quantity of ATP in the sample by the level of ATP per living cell. ATP level in a particular type of viable cell is relatively constant.

The method described in a number of these references and in particular, U.S. Pat. No. 3,745,090, utilizes a non-ionic detergent initially added to the sample to rupture non-bacterial cells placing the ATP from non-bacterial cells in soluble free form. This non-bacterial ATP is then destroyed by adding an ATPase enzyme to hydrolyze the free ATP. The enzyme is destroyed and the bacterial cells are ruptured at the same time freeing their ATP by adding a strong inorganic acid such as nitric acid. the suspension containing free bacterial ATP is neutralized with a buffer and a small amount of luciferase-luciferin reagent along with magnesium chloride is added. The ATP sample and luciferase-luciferin mixture are mixed and brought into the presence of a photo-multiplier system where the maximum light intensity of the bioluminescence is measured. This intensity is related to the amount of ATP present which in turn is related to the amount of viable bacterial cells present in the original sample.

In a preferred embodiment, the method of the present invention measures the ATP level in a sample of the live microbial therapeutic preparations and based upon the known readings from standard samples, the amount of organisms in the microbial therapeutic preparations will be determined. Based upon this measurement, the volume of the suspension of organisms or the dosage levels of the microbial therapeutic preparations may be adjusted to provide the optimum number of organism to the animal for the immunization.

As the bioluminescence measurement of ATP is relatively simple, the test can be performed in the field at the time of administration of the microbial therapeutic preparations to accurately determine the actual number of viable organisms in the microbial therapeutic preparations. Thus if the microbial therapeutic preparations has been stored under questionable conditions, the potency of the microbial therapeutic preparations may be accurately determined prior to administration to the animal. If necessary, the dilution of the microbial therapeutic preparation or the amount of the microbial therapeutic preparations given to the animal may be adjusted to ensure that the optimum number of organisms are administered to elicit the desired response.

The present invention also contemplates the provision of a kit for assessing live microbial therapeutic preparations viability in the field. The kit will include the reagents necessary for performing the test for the selectable marker, a means of measuring the result of the test to determine the level of the selectable marker in the test sample and a means for determining the number of organisms present based on the level of the selectable marker.

For the measurement of the ATP levels using bioluminescence, the kit will include the test reagent for dilution of the sample, an enzyme reagent, and either a standard curve of the plot of relative light units to viable organisms or a table of the ranges of values and the further handling of the preparation depending upon within which range the determined value falls. To perform the test, a suitable luminometer is utilized to measure the relative light units. To compensate for variations in the luminometer utilized in the test, a standard control test sample may be provided with a known level of ATP to standardize the luminometer.

The present invention will now be illustrated by way of examples, but the invention is not limited thereto.

### EXAMPLE 1

The usefulness of the present invention was initially assessed for measuring viable counts of mixed fecal cultures from poultry using ATP bioluminescence as mixed fecal cultures are utilized as the staring materials for preparation of some probiotics. Fecal samples were collected from three 1 week old chicks over a 2 hour period, the samples were pooled together (3 g) and suspended in 20 ml saline. The suspension was blended on a stir plate for 10 min and the heavier particles allowed to settle for 10 min, after which time the supernatant was inoculated into saline at dilutions ranging from 1:5 to 1:100. Colony counts of each suspension was performed using CDC anaerobic blood agar plates. All tubes and plates were incubated at 37° C. in an anaerobic jar. At 1, 4, 7, and 11 days of incubation, the ATP content of the suspensions were measured and compared to the colony count for the suspension.

Microbial ATP was measured using a Raw Milk Bacteria Kit (BIOTRACE™). 200 μl of suspension was mixed with 200 μl of reagent and shaken. Then 100 μl enzyme was added and the light emitted by the reaction was measured about 5 seconds after the reaction commenced on a Uni-Lite XCEL Luminometer (BIOTRACE™) to give Relative Light Units(RLU) which is proportional to the amount of ATP present which in turn is indicative of the number of viable cells present in the sample.

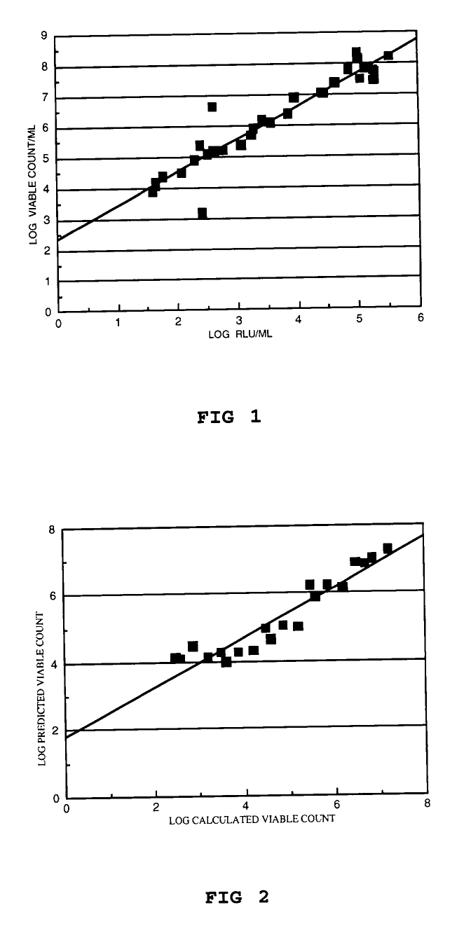

The viable counts (VC) and RLU for the samples from day 0 to day 7 was used to plot the X-Y scatter plot shown in FIG. 1. There was a good correlation between the viable counts and the RLU with an R-square of 0.89080 for the 31 samples plotted. It was found that the minimal bacterial cell number for an accurate estimation using RLU measurements was about 10⁴ organisms/ml. Below this number, the results of the measurements showed increased variability.

### EXAMPLE 2

The RLU values obtained from the 11 day old fecal culture was used to ‘predict’ corresponding viable counts on day 11, using the graph of FIG. 1. To assess the extent of correlation between the predicted and calculated (actual counts) viable counts, log predicted VC Vs. log calculated VC was plotted where Log calculated VC refers to the actual viable counts obtained from day 11 cultures on CDC plates and Log predicted VC refers to the predicted viable counts obtained by extrapolating the RLU values to log viable counts using the graph of FIG. 1. The correlation is shown in FIG. 2 for 20 samples. As shown in FIG. 2, there was a good correlation between the predicted and actual viable count with an R-square of 0.89422.

### EXAMPLE 3

The above two examples were repeated using a commercially available competitive exclusion product. The product chosen was BROILACT™, a mixed culture for prevention of Salmonella infections in chickens. 0.5 g of BROILACT was suspended in 5 ml of saline, mixed well, allowed to settle for 10 minutes and the supernatant was plated for viable counts on CDC anaerobic blood agar plates. The saline suspension was stored at 37° C. and viable counts were measured at different time points. Microbial ATP was measured using the Raw Milk Bacteria Kit (BIOTRACE™) as in Example 1. The readings for RLU were taken on the Uni-Lite XCEL Luminometer (BIOTRAC™) immediately after addition of the enzyme solution. There was a good correlation between the RLU and VC with an R-square value of 0.91911 for the 20 samples plotted.

The RLU values from the above samples were used with the graph of FIG. 1 to extrapolate a predicted viable count. This predicted viable count was then compared with the actual viable count from the plated samples. There was a good correlation found with an R-square value of 0.91803.

### EXAMPLE 4

The method of the present invention was also utilized to determine the ATP content of aged live vaccines. A live poultry coccidiosis vaccine which contained a mixture of Oocysts of the following species: Eimeria tenella, E. necatrix, E. acervulina, E. maxima was used. Similar to the above examples, the ATP content was measured using the ATP Bioluminescence kit from Biotrace™, UK, according to the manufacturer's instructions. The light emitted by the luciferase/luciferin reaction was measured as Relative Light Units (RLU) on a Uni-Lite XCEL Luminometer (Biotrace™, UK). For the Eimeria oocysts, RLU was determined from readings taken between 10 to 20 minutes (sitting time) after sample and reagents were mixed. The representative RLU values of Oocysts taken on samples of the vaccine which had been stored for 1.5, 4.5 and 9.5 months at 4° C. are shown in Tables 1, 2 and 3 respectively. The total number of Oocysts in each sample was determined by a count of representative samples of the Oocyst preparations.

TABLE 1
Age of oocysts: 1.5 months

| | Number of | Sitting | | |
|---|---|---|---|---|
| | oocysts in | time | ATP level | RLU / |
| Sample | the sample | (min.) | (RLU) | oocyst |
| A | 235000 | 10 | 899017 | 3.82 |
| | 235000 | 20 | 958151 | 4.07 |
| B | 304000 | 10 | 1293315 | 4.25 |
| | 304000 | 20 | 1147561 | 3.77 |
| C | 332000 | 10 | 1657673 | 4.99 |
| | 332000 | 20 | 1803645 | 5.43 |
| | | | Average | 4.38 |
| | | | Ratio | |
| | | | Standard | 0.67 |
| | | | Deviation | |

TABLE 2
Age of oocysts: 4.5 months

| | Number of | Sitting | | |
|---|---|---|---|---|
| | oocysts in | time | ATP level | RLU / |
| Sample | the sample | (min.) | (RLU) | oocyst |
| A | 215000 | 10 | 341741 | 1.58 |
| | 215000 | 20 | 322080 | 1.49 |
| B | 218000 | 10 | 371905 | 1.70 |
| | 218000 | 20 | 361552 | 1.65 |
| C | 243000 | 10 | 367077 | 1.51 |
| | 243000 | 20 | 461223 | 1.89 |
| | | | Average | 1.63 |
| | | | Ratio | |
| | | | Standard | 0.14 |
| | | | Deviation | |

TABLE 3
Age of oocysts: 9.5 months

| | Number of | Sitting | | |
|---|---|---|---|---|
| | oocysts in | time | ATP level | RLU / |
| Sample | the sample | (min.) | (RLU) | oocyst |
| A | 200666 | 10 | 176619 | 0.88 |
| | 200666 | 20 | 178276 | 0.88 |
| B | 81000 | 10 | 99776 | 1.23 |
| | 81000 | 20 | 106113 | 1.31 |
| C | 171000 | 10 | 220241 | 1.28 |
| | 171000 | 20 | 219881 | 1.28 |
| | | | Average | 1.14 |
| | | | Ratio | |
| | | | Standard | 0.20 |
| | | | Deviation | |

Table 4 summarizes the Average RLU/oocyst for the three aged samples.

TABLE 4
Decrease in RLU of oocysts with aging:

| Age in | Average RLU/ |
|---|---|
| months | oocyst |
| 1.5 | 4.4 |
| 4.5 | 1.6 |
| 9.5 | 1.1 |

A corresponding decline in infectivity in chicks of the three aged samples was also observed, indicating that the levels of ATP in a coccidiosis vaccine is an accurate indicator of the number of viable, infective Oocysts in the vaccine.

The results of the above clearly demonstrate that bioluminescence measurements are applicable to a rapid estimation of microbial numbers in a live microbial preparation such as a mixed bacterial culture used as a probiotic, and for the assessment of the viability/infectivity of Eimeria oocysts in a vaccine preparation. The experiments demonstrate a high overall correlation and linearity between RLU values and viable counts. Measuring RLU values is a quicker method (about 30 to 40 sec per suspension) for the estimation of viability compared to measuring actual viable plate counts for bacteria(takes 3 days of incubation on an average), or lesion scores for Eimeria (5 to 7 days to complete in chicks).

The present invention permits a rapid estimation of viable cell numbers in a live microbial product. This method offers a means of quality control of a live culture especially when cell numbers could decrease on storage or by improper handling thereby adversely affecting the use of the product.

The present invention may be easily practiced in the field or as part of the quality control procedures in the manufacture of a live microbial preparation. Many such preparations, especially probiotics which generally contain anaerobic bacteria as provided to the end user as a lyophilized preparation. The user would reconstitute the preparation, following the manufacturer's instructions and a sample of the reconstituted preparation would be assayed for the amount of the selectable marker, such as the level of ATP in the preparation. The manufacturer could provide a standard curve showing the level of the selectable marker to the number of viable organisms in the preparation. From the reading, the number of the viable organisms in the preparation could be determined and based upon this number, instructions for further handling of the preparation to optimize the therapy could be given. These instructions could include further dilution of the preparation or could include recommended dosage levels based upon the number of viable organisms in the preparation. Alternatively, the manufacturer of the live microbial therapeutic preparation could provide instructions for further handling based upon the level of the selectable marker without having to extrapolate the number of viable organisms in the preparation. For example, based upon the RLU reading from an ATP assay, the user could be instructed to further dilute the preparation if the reading was within a specified range, or they could be given instructions regarding the dosage levels to be administered depending upon within which range of values the measured value fell. In order to provide a confirmation of the accuracy of the test a single standard would be run. If the value of the standard were within an specified range, the test would be accurate. If the standard were not within the range, instructions could be provided to enable a user to adjust the value of the test sample prior to following the further instructions.

The present invention is also usable in quality control of manufacture of live microbial therapeutic preparations. The method of the present invention can accurately determine the number of viable organism in a preparation at the time of manufacture so that the preparation my be properly aliquoted to the optimal dosage levels. The method of the present invention can also be utilized to provide a accurate determination of the expected number of viable organism remaining on varying storage conditions of the preparation. Representative samples of the preparations may be store under varying time, temperature and other environmental conditions and based upon the level of the selectable marker, the number of viable organisms may be accurately determined. This could aid in easily determining whether a preparation would be still usable if it were stored under those conditions by the end user prior to the administration of the preparation.

Live vaccines play a crucial role in eliciting protective immunity for the prevention of a variety of poultry diseases. The titre and the infectivity of the vaccine is important in determining its protective capacity. In the case of coccidiosis, the viability of Eimeria oocysts determines the potency of the vaccine. As the Oocysts age in cold storage, there is a concomitant decrease in its potency/infectivity. At present the viability of Eimeria oocysts is determined via their infectivity or lesion scores in chicks, a process which requires five to seven days.

In a preferred embodiment, the present invention provides a simple and accurate method of determining the viability of organisms in a live vaccine and in particular, the viability of Eimeria oocysts in a live coccidiosis vaccine in the field by the measurement of the ATP levels using bioluminescence. Once the number of organisms are determined, the dosage level of the vaccine may be adjusted to provide the optimum number of organisms to the animal for the purpose of immunizing the animal against the disease.

The present invention overcomes the problems associated with storage of the microbial therapeutic preparations, where even under recommended conditions, the viability of the organisms gradually decreases. If the microbial therapeutic preparations is stored under potentially detrimental conditions or if stored for long periods of time, the viability of the organisms may decrease to a level where a standard dose in accordance with the original instructions for use may not provide adequate protection against the disease state. This is particularly important for microbial therapeutic preparations which utilize low numbers of organisms or where the storage conditions may not be ideal. By using the method of the present invention, the number of viable organisms in the microbial therapeutic preparations may be accurately determined just prior to administration of the microbial therapeutic preparations and the dosage adjusted if necessary.

The method of the present invention is also useful in quality control for microbial therapeutic preparations manufacture. When a microbial therapeutic preparations is being formulated, it is necessary to know the number of organisms which have been produced so that accurate doses of microbial therapeutic preparations may be prepared. With the presently utilized colony count techniques and other methods, the time to complete the assay may be significant and the viability of the preparation may decrease. The method of the present invention provides an accurate and rapid determination of the number of viable organisms in a preparation so that the time between the production of the organism preparation and the dispensing of microbial therapeutic preparations doses is greatly reduced.

Although various preferred embodiments of the present invention have been described herein in detail, it will be appreciated by those skilled in the art, that variations may be made thereto without departing from the spirit of the invention or the scope of the appended claims.

The embodiments of the invention in which an exclusive property or privilege is claimed are defined as follows:

1. A method of assessing the viability of organisms in a live microbial therapeutic preparation comprising:
a) measuring a suspension of the organisms for the quantity of a selectable marker indicative of viable organisms; and
b) comparing the measurements of step a) against a predetermined standard to give a count of the viable organisms in the suspension.

2. A method as claimed in claim 1 wherein the live microbial therapeutic preparation is a probiotic or a live attenuated vaccine.

3. A method as claimed in claim 2 wherein the selectable marker is the level of ATP in the suspension of organisms.

4. A method as claimed in claim 3 wherein the predetermined standard is a standard curve of ATP levels versus viable organisms in the preparation

5. A method of assessing the viability of Eimeria oocysts in a live coccidiosis vaccine comprising:
a) measuring a suspension of the Eimeria oocysts for the quantity of ATP bioluminescence in the suspension; and
b) comparing the quantity of ATP bioluminescence in the suspension of step a) against a predetermined standard curve to give a count of the viable Eimeria oocysts in the live coccidiosis vaccine.

6. A method as claimed in claim 5 wherein the predetermined standard is a standard curve of ATP levels versus viable organisms in the preparation

7. A kit for assessing the number of viable organisms in a live microbial therapeutic preparation in the field, the kit comprising reagents necessary for performing the test for a selectable marker indicative of viable organisms, a means of measuring the result of the test to determine the level of the selectable marker in the test sample and a means for determining the number of viable organisms present based on the level of the selectable marker.

8. A kit as claimed in claim 7 wherein the live microbial therapeutic preparation is a probiotic or a live attenuated vaccine.

9. A kit as claimed in claim 8 wherein the selectable marker is the level of ATP in the suspension of organisms.

10. A kit as claimed in claim 9 wherein the means for determining the number of viable organisms present based on the level of the selectable marker is a standard curve of ATP levels versus viable organisms in the preparation.

11. A kit for assessing viability of Eimeria oocysts in a live coccidiosis vaccine in the field by the measurement of the ATP levels using bioluminescence, the kit comprising test reagent for dilution of the sample, an enzyme reagent, and a means for determining the number of viable organisms present based on the ATP level.

12. A kit as claimed in claim 11 wherein the means for determining the number of viable organisms present based on the ATP levels a standard curve of the plot of relative light units emitted from a test sample to viable organisms.

13. A kit as claimed in claim 12 further comprising a suitable luminometer to measure the relative light units.

14. A kit as claimed in claim 13 further including a standard control test sample containing a known level of ATP.
